(19) United States
(12) Patent Application Publication
(10) Pub. No.: US 2020/0008977 A1
(43) Pub. Date: Jan. 9, 2020

(54) DEVICES FOR DUAL MECHANISM AESTHETIC TREATMENT AND METHODS OF USE THEREOF

(71) Applicant: VIVEVE, INC., Englewood, CO (US)

(72) Inventors: James ATKINSON, Greenwood Village, CO (US); David BLACK, Cameron, MT (US); Richard HATCH, Pleasanton, CA (US); Patricia SCHELLER, Solebury, PA (US); Perry TOMASETTI, Pepperell, MA (US); Deborah WILKERSON, Denver, CO (US)

(21) Appl. No.: 16/482,581

(22) PCT Filed: Jan. 31, 2018
(86) PCT No.: PCT/US2018/016266

§ 371 Date: Jul. 31, 2019

Related U.S. Application Data
(60) Provisional application No. 62/452,889, filed on Jan. 31, 2017.

(51) Int. Cl. A61F 7/12; A61F 7/00; A61N 1/40; A61N 7/02; A61N 5/02

(52) U.S. Cl. CPC A61F 7/12; A61F 7/0085; A61N 1/403; A61N 7/022; A61N 5/025; A61F 2007/0296; A61F 2007/0017; A61F 2007/0006; A61F 2007/0028; A61F 2007/001; A61F 2007/0052; A61F 2007/005

(57) ABSTRACT

The systems and methods in accordance with the principles of the invention can promote correction of an aesthetic or functional defect in a target tissue. A method can include non-invasively applying a cooling agent to a surface of a target tissue; and cooling one or more tissue layers of said target tissue to a predetermined therapeutic temperature, wherein applying the cooling agent is performed such that cryoablation of said one or more layers of the target tissue does not occur. The system can include: a controller and a probe having a distal end configured for non-invasive contact with a surface of a target tissue to cool the target tissue based on treatment parameters.

### CROSS-REFERENCE OF RELATED APPLICATION

This application claims priority to U.S. Provisional Patent Application No. 62/452,889, filed on Jan. 31, 2017; the entire contents of which are hereby incorporated by reference.

### BACKGROUND

### 1. Technical Field

The field of the currently claimed embodiments of this invention relates to a method and apparatus for promoting correction of an aesthetic or functional defect in a target tissue.

### 2. Discussion of Related Art

Wound healing is the process by which skin or other body tissue repairs itself after trauma. In undamaged skin, the epidermis (surface layer) and dermis (deeper layer) form a protective barrier against the external environment. When the barrier is broken, an orchestrated cascade of biochemical events is set into motion to repair the damage. This process is divided into predictable phases: blood clotting (hemostasis), inflammation, tissue growth (proliferation) and tissue remodeling (maturation).

Tissue remodeling begins when the levels of collagen production and degradation equalize. During this stage, type III collagen is replaced by type I collagen. Originally disorganized collagen fibers are rearranged, cross-linked, and aligned along tension lines. The onset of the maturation phase may vary extensively, depending on the size of the wound and whether it was initially closed or left open. Tissue remodeling can last for a year or longer, similarly depending on wound type.

There remains a need for a non-invasive, targeted and therapeutic approach for activating remodeling in a wound to better promote wound healing and reduce the formation of scar tissue.

### SUMMARY

Some embodiments of the invention relate to a non-invasive probe for promoting correction of an aesthetic or functional defect in a target tissue, having a treatment tip configured for non-invasive contact with a surface of a target tissue. The treatment tip includes: an epithelium-contacting treatment surface; a cooling element in thermal communication with the epithelium-contacting treatment surface; and a heating element in thermal communication with the epithelium-contacting treatment surface. The non-invasive probe also has a controller in communication with the cooling element and the heating element. The controller is configured to control the cooling element to cool the epithelium-contacting treatment surface to a predetermined temperature. The controller is also configured to control the cooling element and the heating element to maintain the predetermined temperature for a predetermined period of time to induce wound healing in the target tissue.

Some embodiments of the invention relate to the non-invasive probe above, where the controller is configured to control the cooling element and the heating element to cool or heat a first portion of the epithelium-contacting treatment surface while simultaneously heating or cooling a second portion of the epithelium-contacting treatment surface, and where the epithelium-contacting treatment surface is radially oriented.

Some embodiments of the invention relate to the non-invasive probe above, where the epithelium-contacting treatment surface has a length of between 1 mm and 30 mm and a width of between 0.5 cm and 2.0 cm.

Some embodiments of the invention relate to the non-invasive probe above, where the controller is configured to activate the cooling element for a first period of time and to activate the heating element for a second period of time, such that the first period of time overlaps at least partially with the second period of time.

Some embodiments of the invention relate to the non-invasive probe above, where the controller is configured to activate the cooling element for a first period of time and to activate the heating element for a second period of time, such that the first period of time commences and ends prior to commencement of the second period of time.

Some embodiments of the invention relate to the non-invasive probe above, where the heating element is a bipolar radiofrequency (RF) energy heating element.

Some embodiments of the invention relate to a non-invasive system for promoting correction of an aesthetic or functional defect in a target tissue, the system having: a controller coupled to a probe for promoting wound healing in the target tissue, the probe having a distal end configured for non-invasive contact with a surface of the target tissue and having a proximal end coupled to the controller; and at least one aesthetic or functional defect treatment parameter the at least one aesthetic or functional defect treatment parameter selected to achieve a predetermined temperature for a predetermined time period in a target tissue to induce remodeling of the target tissue and promote wound healing. The controller coupled to the probe is configured to cool the target tissue based on the at least one aesthetic or functional defect treatment parameter to induce a remodeling of the target tissue for improvement of the aesthetic or functional defect.

Some embodiments of the invention relate to the non-invasive system above, further including the probe, wherein the probe includes: a treatment tip configured for non-invasive contact with a surface of a target tissue, the treatment tip including: an epithelium-contacting treatment surface; a cooling element in thermal communication with the epithelium-contacting treatment surface; and a heating element in thermal communication with the epithelium-contacting treatment surface. The controller is further configured to be in communication with the cooling element and the heating element, and the controller is further configured to control the cooling element and the heating element to cool or heat a first portion of the epithelium-contacting treatment surface while simultaneously heating or cooling a second portion of the epithelium-contacting treatment surface.

Some embodiments of the invention relate to the non-invasive system above, where the epithelium-contacting treatment surface is radially oriented.

Some embodiments of the invention relate to the non-invasive system above, where the epithelium-contacting treatment surface has a length of between 1 mm and 30 mm and a width of between 0.5 cm and 2.0 cm.

Some embodiments of the invention relate to the non-invasive system above, where the controller is further configured to activate the cooling element for a first period of time and to activate the heating element for a second period of time, such that the first period of time overlaps at least partially with the second period of time.

Some embodiments of the invention relate to the non-invasive system above, where the controller is further configured to activate the cooling element for a first period of time and to activate the heating element for a second period of time, such that the first period of time commences and ends prior to commencement of the second period of time.

Some embodiments of the invention relate to the non-invasive system above, where the cooling element is configured to apply a cooling agent to the treatment tip, and the cooling agent is one or more of compressed liquid N₂, compressed liquid N₂, compressed liquid CO₂, compressed liquid NO₂, a hydrofluorocarbon, water, a thermoelectric cooler and an ultra-low temperature cryogen.

Some embodiments of the invention relate to the non-invasive system above, where the heating element is a bipolar radiofrequency (RF) energy heating element.

Some embodiments of the invention relate a method for aesthetic treatment, including the steps: non-invasively cooling a surface of a target tissue; and cooling one or more tissue layers of the target tissue to a predetermined therapeutic temperature. The step of non-invasively cooling is performed such that cryoablation of the one or more tissue layers of the target tissue does not occur.

Some embodiments of the invention relate the method above, further including: non-invasively heating the surface of the target tissue; and heating the one or more tissue layers of the target tissue to maintain a temperature of the one or more tissue layers above a temperature at which cryoablation occurs.

Some embodiments of the invention relate the method above, where the non-invasively cooling is performed over a first period of time, the non-invasively heating is performed over a second period of time, and the first period of time overlaps at least partially with the second period of time.

Some embodiments of the invention relate the method above, where the non-invasively cooling commences before the non-invasively heating commences and where the non-invasively heating continues until the non-invasively cooling is terminated.

Some embodiments of the invention relate the method above, where the non-invasively heating occurs concurrently with the non-invasively cooling.

Some embodiments of the invention relate the method above, where the non-invasively heating includes non-invasively applying a heating agent and delivering at least one of radiofrequency energy, microwave energy, laser energy, or ultrasound energy.

Some embodiments of the invention relate the method above where the target tissue includes female genital tissue.

Some embodiments of the invention relate the method above where the target tissue includes tissues of the anus, anal canal and/or rectum.

Some embodiments of the invention relate the method above where the cooling involves cooling the one or more tissue layers to a temperature between 1.1 degrees Celsius and 4.0 degrees Celsius and where the cooling triggers a wound-healing reaction in the one or more tissue layers of the target tissue.

Some embodiments of the invention relate the method above, where the non-invasively cooling includes contacting the one or more tissue layers of the target tissue with a treatment tip during a procedure, the treatment tip including a cooling mechanism.

Some embodiments of the invention relate the method above, further including contacting the one or more tissue layers with the treatment tip at two or more contact sites during the procedure and where the contacting the one or more tissue layers is repeated at least twice during the procedure such that each of the two or more contact sites is contacted at least twice.

Some embodiments of the invention relate the method above, where the non-invasively cooling includes evaporating compressed liquid N₂, CO₂, or NO₂ on a surface of the treatment tip and contacting the surface of the target tissue with the treatment tip.

Some embodiments of the invention relate the method above, where the cooling the one or more tissue layers of the target tissue induces a remodeling of the one or more tissue layers, where the remodeling involves one or more of a release of heat shock proteins and a release of cold shock proteins, and where at least some of the remodeling occurs during the cooling of the one or more tissue layers of the target tissue.

Some embodiments of the invention relate the method above, where the non-invasively applying a cooling agent is done for between 1 second to 300 seconds.

Some embodiments of the invention relate the method above further including the step of treating an aesthetic injury or a functional defect in a subject.

Some embodiments of the invention relate the method above further including the step of treating one or more of a vaginal mucosa, an oral mucosa, a naso-pharyngeal mucosa, an esophageal mucosa, a rectal mucosa or an anal mucosa.

### BRIEF DESCRIPTION OF THE DRAWINGS

Further objectives and advantages will become apparent from a consideration of the description, drawings, and examples.

FIG. 1A illustrates an apparatus for applying a cooling agent and/or a heating agent to a target tissue.

FIG. 1B is an illustration of an apparatus for applying a cooling agent and/or a heating agent to the target tissue, wherein the treatment surface extends to the proximal portion of the apparatus.

FIG. 2 is a schematic view of female genitalia, as well as an orienting clock to provide a circumferential reference scheme for the vaginal wall.

FIG. 3A is a schematic illustration of three locations for the application of pulses by a treatment tip on the surface of a target tissue according to some embodiments of the invention, where the three locations are adjacent to one another.

FIG. 3B is a schematic illustration of three locations for the application of pulses by a treatment tip on the surface of a target tissue according to some embodiments of the invention, where the three locations overlap at least partially with one another.

FIG. 3C is a schematic illustration of three locations for the application of pulses by a treatment tip on the surface of a target tissue according to some embodiments of the invention, where the three locations are separated from each other by a predetermined distance.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Non-invasive, targeted and therapeutic approaches are provided for activating remodeling in a wound and/or tissue to better promote wound healing and/or reduce the formation of scar tissue. The devices, systems and methods provide a minimally invasive way to treat, for example, aesthetic injuries or flaws of a target tissue.

Heat shock proteins (HSPs) and cold shock proteins (CSPs) are families of proteins that are produced by cells in response to exposure to stressful conditions. HSPs were first described in relation to heat shock, but are now known to also be expressed during other stresses including exposure to cold, UV light, and during wound healing or tissue remodeling. Indeed, HSPs and the various biological processes they are associated with are recognized to be active players in tissue remodeling.

CSPs are proteins having a cold-shock domain (CSD) of about 70 amino acids which has been found in prokaryotic and eukaryotic DNA-binding proteins. Part of this domain is highly similar to the RNP-1 RNA-binding motif. CSPs are expressed in a cell or tissue when temperatures fall below that cell or tissue's normal temperature. For instance, when Escherichia coli is exposed to a temperature drop from 37 to 10 degrees Celsius, a 4-5 hour lag phase occurs, after which growth is resumed at a reduced rate. During the lag phase, the expression of around 13 proteins, which contain cold shock domains is increased 2-10 fold. These so-called “cold shock” proteins are thought to help the cell to survive in temperatures lower than optimum growth temperature, by contrast with heat shock proteins, which help the cell to survive in temperatures greater than the optimum, possibly by condensation of the chromosome and organization of the prokaryotic nucleoid. Although the role of CSPs in tissue remodeling is unclear, it is clear that these proteins have an effect on the biological processes of cooled cells and tissues and a role for CSPs in tissue remodeling might exist.

HSPs in a wounded tissue can be stimulated by exposing the tissue to cold or heat. Unfortunately, care has to be taken to avoid ablation of the tissues as a result of extremely cold or hot temperatures as occurs during cryoablation or catheter ablation, respectively. This is not desirable when seeking to promote wound healing and limiting the formation of scar tissue and improving the aesthetic appearance of a wound once it heals.

Cryoablation and catheter ablation result in the destruction of tissue. Traditional methods and devices for cryo or thermal ablation result in the destruction of tissues. For example, during cryoablation, hollow needles (cryoprobes) are used to contact and cool target tissues to temperatures below freezing. These cryoprobes are cooled by circulating cooled, thermally conductive fluids within them. Cryablation ultimately leads to apoptosis of cells within a target tissue, resulting in the destruction of regions within the target tissue. During thermal ablation, a catheter is used to contact and deliver a heating source to a target tissue, resulting in heating of the tissue to a temperature sufficiently high enough to cause destruction of the tissue. In short, traditional methods and devices for cryo and thermal ablation are invasive and result in the destruction of target tissues. By contrast, devices described herein (and methods of using such devices) are designed to be minimally invasive and to avoid or minimize tissue destruction.

Apparatus and System

Some embodiments of the invention relate to a non-invasive probe for promoting correction of an aesthetic or functional defect in a target tissue, having a treatment tip configured for non-invasive contact with a surface of a target tissue. The treatment tip includes: an epithelium-contacting treatment surface; a cooling element in thermal communication with the epithelium-contacting treatment surface; and a heating element in thermal communication with the epithelium-contacting treatment surface. The non-invasive probe also has a controller in communication with the cooling element and the heating element. The controller is configured to control the cooling element to cool the epithelium-contacting treatment surface to a predetermined temperature. The controller is also configured to control the cooling element and the heating element to maintain the predetermined temperature for a predetermined period of time to induce wound healing in the target tissue.

Some embodiments of the invention relate to the non-invasive probe above, where the controller is configured to control the cooling element and the heating element to cool or heat a first portion of the epithelium-contacting treatment surface while simultaneously heating or cooling a second portion of the epithelium-contacting treatment surface, and where the epithelium-contacting treatment surface is radially oriented.

Some embodiments of the invention relate to the non-invasive probe above, where the epithelium-contacting treatment surface has a length of between 1 mm and 30 mm and a width of between 0.5 cm and 2.0 cm.

Some embodiments of the invention relate to the non-invasive probe above, where the controller is configured to activate the cooling element for a first period of time and to activate the heating element for a second period of time, such that the first period of time overlaps at least partially with the second period of time.

Some embodiments of the invention relate to the non-invasive probe above, where the controller is configured to activate the cooling element for a first period of time and to activate the heating element for a second period of time, such that the first period of time commences and ends prior to commencement of the second period of time.

Some embodiments of the invention relate to the non-invasive probe above, where the heating element is a bipolar radiofrequency (RF) energy heating element.

Some embodiments of the invention relate to a probe for promoting correction of an aesthetic or functional defect in a target tissue. In such embodiments, the probe includes a treatment tip configured for non-invasive contact with a surface of a target tissue. The treatment tip includes an epithelium-contacting treatment surface, a cooling element in thermal communication with the epithelium-contacting treatment surface, and a bipolar radiofrequency (RF) energy heating element in thermal communication with the epithelium-contacting treatment surface. In such embodiments, the probe also includes a controller in communication with the cooling element and the bipolar RF energy heating element. The controller is configured to control the cooling element to cool the epithelium-contacting treatment surface to a predetermined temperature. The controller is also configured to control the cooling element and the bipolar RF energy heating element to maintain the predetermined temperature for a predetermined period of time to induce wound healing in the target tissue.

Some embodiments of the invention relate to the probe described above, where the controller is configured to control the cooling element and the heating element to cool or heat a first portion of the epithelium-contacting treatment surface while simultaneously heating or cooling a second portion of the epithelium-contacting treatment surface.

Some embodiments of the invention relate to the probe described above, where the epithelium-contacting treatment surface is radially oriented. In such embodiments, the term “radially oriented” relates to having a curved treatment surface such that the curved treatment surface is designed to interface with a curved tissue surface. The curved treatment surface can be concave so that it interacts with a convex tissues surface, or convex so that it interacts with a concave tissue surface. In some embodiments, the probe has two or more treatment surfaces. In such embodiments, one or more of the treatment surfaces can be radially oriented. Also, in such embodiments the various treatment surfaces can have varying degrees of curvature. For example, in some such embodiments, a first treatment surface can be convex and a second treatment surface can be concave. In some embodiments, the probe has two or more treatment surfaces that are flat, but are oriented at different angles with respect to the handle of the probe such that the flat treatment surfaces can come into contact with different portions of a curved tissue surface.

Some embodiments of the invention relate to the probe described above, where epithelium-contacting treatment surface has a length between 1 mm and 30 mm and a width between 0.5 cm and 2.0 cm.

Some embodiments of the invention relate to the probe described above, where the controller is configured to activate the cooling element for a first period of time and to activate the heating element for a second period of time, such that the first period of time overlaps at least partially with the second period of time.

Some embodiments of the invention relate to the probe described above, where the controller is configured to activate the cooling element for a first period of time and to activate the heating element for a second period of time, such that the first period of time commences and ends prior to commencement of the second period of time.

Some embodiments of the invention relate to the probe described above, where the cooling element is configured to apply cooling to a surface of the target tissue. Applying cooling to the tissue can take various forms. For example, in one aspect the cooling is provided to the tissue by way of an applicator, such as a tip, and the cryogen is applied to the applicator or tip and the applicator or tip contacts the tissue. The cryogen can be applied inside the applicator to chill the tip or the back of an electrode(s). Preferably with mucosal tissue, the cryogen is not applied directly to the tissue but rather through the applicator.

Some embodiments of the invention relate to the probe described above, where the cooling agent is selected from the following: compressed liquid N₂, compressed liquid CO₂, compressed liquid NO₂, a hydrofluorocarbon, water, a thermoelectric cooler and an ultra-low temperature cryogen.

Cooling and applying cooling in accordance with the principles of the invention can include cooling using a treatment tip that applies cooling through contact, such as applying a cooled treatment tip, applying a cooling agent and/or composition, including directly and indirectly to the tissue or surface, and all of these modalities for applying cooling can be referred to as applying cooling, a cooling agent or otherwise, interchangeably for purposes of this disclosure, although each modality may have advantages relative to the others. As indicated above, with mucosal tissue it is preferred to apply cooling to the tissue by way of an intermediary, such as a chilled tip, and not applying the cryogen material directly to the tissue. Cooling can be applied to the tissue sequentially with heating and/or simultaneously with heating.

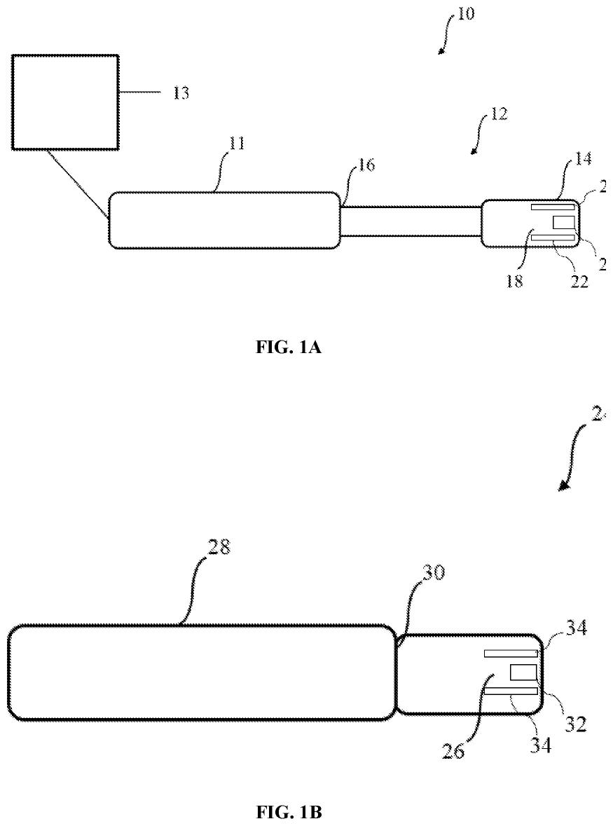

Exemplary probes are shown in FIGS. 1A and 1B, and modifications can be made in order to treat the target tissue effectively and efficiently. FIG. 1A shows an exemplary treatment device 10 for promoting correction of an aesthetic or functional defect in a target tissue. A probe is shown generally at reference numeral 12 to promote correction of an aesthetic or functional defect in a target tissue. The probe 12 has a treatment tip 14 configured for non-invasive contact with a surface of a target tissue, and has a proximal end 11 coupled by an arm 16 to the distal end 14. The treatment tip includes an epithelium-contacting treatment surface 18, a cooling element 20 in thermal communication with the epithelium-contacting treatment surface 18 and a bipolar RF energy heating element 22 in thermal communication with the epithelium-contacting treatment surface 18. One of skill in the art can envision that the cooling element 20 and the heating element 22 are arranged in varying configurations and can also be positioned at various distances from one another. In some cases, the cooling and heating elements are proximate to one another. In other cases they can be overlapping. Yet in other cases they can be separated by a desired distance. In accordance with the principles of the inventions herein, a controller 13 in communication with the probe controls the parameters for the treatment of at least one aesthetic or functional defect. At least one aesthetic or functional defect treatment parameter is selected to achieve a predetermined temperature for a predetermined time period in a target tissue to induce remodeling of the target tissue and promote wound healing. The controller coupled to the probe is configured to cool the target tissue based on the at least one aesthetic or functional defect treatment parameter to induce a remodeling of the target tissue for improvement of the aesthetic or functional defect. The distal end 14 of probe 12 has a treatment surface 18 for contacting the target tissue. In some embodiments, the controller 13 can be disposed within the proximal end 11 of the probe 12. In some embodiments, the proximal end 11 of the probe 12 may form a handle to allow a user to hold and manipulate the probe 12 during treatment.

As shown in FIG. 1A, the treatment surface 18 according to some embodiments comprises a single treatment surface. The treatment surface 18 may be flat, or may be curved or angled to achieve better contact with the surface of the target tissue. In some embodiments, the treatment surface, which comes into contact with surface regions of the target tissues, may have a total surface area between about 0.5 cm² and 6 cm². The total length of the treatment surface area can be between about 1 mm and 30 mm (or between 0.1 cm and 3 cm), and the total width can be between about 0.5 cm and 2.0 cm. One of skill in the art may contemplate other lengths and widths that are appropriate for treating specific target tissues, and would understand the embodiments of the invention to include devices having these configurations.

A probe 24 according to some embodiments is shown in FIG. 1B. The probe 24 includes an epithelium-contacting treatment surface 26 that extends all the way to the proximal portion 28 of the probe 24. The treatment surface 26 may be directly coupled to the proximal portion 28 at the proximal end 30 of the treatment surface 26, or may be joined to the proximal portion 28 by a connecting arm, as shown in FIG. 1A. The treatment tip includes a cooling element 32 in thermal communication with the epithelium-contacting treatment surface and a heating element 34 in thermal communication with the epithelium-contacting treatment surface. One of skill in the art can envision that the cooling element and the heating element are arranged in varying configurations and can also be positioned at various distances from one another. In some cases, the cooling and heating elements are proximate to one another. In other cases they can be overlapping. Yet in other cases they can be separated by a desired distance. There can also be a plurality of cooling and/or heating elements positioned on the treatment surface. The treatment surface 26 may have a total surface area between about 0.5 cm² and 6 cm². The total length of the treatment surface can be between about 0.1 cm and about 6 cm. The total width of the treatment surface according to some embodiments can be between about 0.5 cm and about 3.0 cm. One of skill in the art may contemplate other lengths and widths that are appropriate for treating target tissue, and would understand the embodiments of the invention to include devices having these dimensions.

The probe may further include more than one treatment surface. In such embodiments, the probe can include an adjustment mechanism for drawing the treatment surfaces closer together, or for moving them farther apart. According to some embodiments, the adjustment mechanism can allow the treatment surfaces to be moved such that they are adjacent to one another and form a continuous treatment surface, like the treatment surface 18 in FIG. 1A. The two treatment surfaces may be positioned parallel to one another. Alternatively, the treatment surfaces may be positioned at an angle with respect to one another and to the probe, such that they are better configured to make contact with the surface of the target tissue.

The treatment surfaces may include one or more cooling elements and heating elements. The cooling elements and heating elements may enable cooling and/or heating, respectively, of an entire treatment surface at once. Alternatively, each cooling element or heating element may enable cooling or heating of multiple portions of the treatment surface, individually or simultaneously. Each of the treatment surfaces may also have a plurality of cooling elements and/or heating elements that can cool or heat, respectively, sections of the treatment surface separately and/or in succession. For example, the treatment surface 20 may be divided into a number of sections along its length. Cooling may be applied for a first period of time in the first section, and once the first period of time ends, cooling of the first section may end, while cooling of the second section may begin. This may continue along the length of the treatment tip, until all sections of the treatment surface 20 have undergone cooling. The treatment surface 22 may undergo a similar heating process at the same time, or the processes may be conducted at different times. Multiple treatments can occur at one location. In such instances, heating may precede, follow or occur concurrently with the cooling such that the target tissue is maintained at a desired therapeutic temperature. By heating the tissue, ablation of the tissue due to unintentional freezing of the tissue is prevented. In some embodiments, the target tissue is cooled and no heating is applied. In some embodiments, the heating element is a bipolar radiofrequency energy element.

Further, the treatment surfaces may be configured such that individual sections can undergo cooling. For example, the first section may undergo cooling prior to heating of the same first section. The cooling may cease while the heating of the first section takes place. During this period, the second section may undergo a cooling process. When the heating of the first section ends, cooling of the first section may resume for a period of time, while heating of the next section begins. This process may continue along the length of the treatment surfaces. This process is purely exemplary, and other combinations and patterns of heating and cooling may also be used. The controller 13 may control the cooling elements and bipolar RF energy heating elements to achieve the desired treatment pattern and to ensure that the therapeutic temperature is maintained.

In embodiments having more than one treatment surface, the total surface area of the multiple treatment surfaces can be between about 0.5 cm² and about 6 cm². One of skill in the art may contemplate other surface areas that are appropriate for treating specific target tissues, and would understand the embodiments of the invention to include devices having these configurations.

Some embodiments of the invention relate to a non-invasive system for promoting correction of an aesthetic or functional defect in a target tissue, the system having: a controller coupled to a probe for promoting wound healing in the target tissue, the probe having a distal end configured for non-invasive contact with a surface of the target tissue and having a proximal end coupled to the controller; and at least one aesthetic or functional defect treatment parameter the at least one aesthetic or functional defect treatment parameter selected to achieve a predetermined temperature for a predetermined time period in a target tissue to induce remodeling of the target tissue and promote wound healing. The controller coupled to the probe is configured to cool the target tissue based on the at least one aesthetic or functional defect treatment parameter to induce a remodeling of the target tissue for improvement of the aesthetic or functional defect.

Some embodiments of the invention relate to the non-invasive system above, further including the probe, wherein the probe includes: a treatment tip configured for non-invasive contact with a surface of a target tissue, the treatment tip including: an epithelium-contacting treatment surface; a cooling element in thermal communication with the epithelium-contacting treatment surface; and a heating element in thermal communication with the epithelium-contacting treatment surface. The controller is further configured to be in communication with the cooling element and the heating element, and the controller is further configured to control the cooling element and the heating element to cool or heat a first portion of the epithelium-contacting treatment surface while simultaneously heating or cooling a second portion of the epithelium-contacting treatment surface.

Some embodiments of the invention relate to the non-invasive system above, where the epithelium-contacting treatment surface is radially oriented.

Some embodiments of the invention relate to the non-invasive system above, where the epithelium-contacting treatment surface has a length of between 1 mm and 30 mm and a width of between 0.5 cm and 2.0 cm.

Some embodiments of the invention relate to the non-invasive system above, where the controller is further configured to activate the cooling element for a first period of time and to activate the heating element for a second period of time, such that the first period of time overlaps at least partially with the second period of time.

Some embodiments of the invention relate to the non-invasive system above, where the controller is further configured to activate the cooling element for a first period of time and to activate the heating element for a second period of time, such that the first period of time commences and ends prior to commencement of the second period of time.

Some embodiments of the invention relate to the non-invasive system above, where the cooling element is configured to apply a cooling agent to the treatment tip, and the cooling agent is one or more of compressed liquid N₂, compressed liquid N₂, compressed liquid CO₂, compressed liquid NO₂, a hydrofluorocarbon, water, a thermoelectric cooler and an ultra-low temperature cryogen.

Some embodiments of the invention relate to the non-invasive system above, where the heating element is a bipolar radiofrequency (RF) energy heating element.

Some embodiments of the invention relate to a system for promoting correction of an aesthetic or functional defect in a target tissue. In such embodiments, the system includes a controller coupled to a probe for promoting wound healing in the target tissue. The probe includes a distal end configured for non-invasive contact with a surface of the target tissue and a proximal end coupled to the controller. In such embodiments, the system also includes at least one aesthetic or functional defect treatment parameter. The at least one aesthetic or functional defect treatment parameter is selected to achieve a predetermined temperature for a predetermined time period in a target tissue to induce remodeling of the target tissue and promote wound healing. In such embodiments, the controller coupled to the probe is configured to cool the target tissue based on the at least one aesthetic or functional defect treatment parameter to induce a remodeling of the target tissue for improvement of the aesthetic or functional defect.

Some embodiments of the invention relate to the system described above, where the system further includes the probe. In such embodiments, the probe includes a treatment tip configured for non-invasive contact with a surface of a target tissue. The treatment tip includes an epithelium-contacting treatment surface a cooling element in thermal communication with the epithelium-contacting treatment surface, and heating element in thermal communication with the epithelium-contacting treatment surface. In such embodiments, the controller is further configured to be in communication with the cooling element and heating element.

Some embodiments of the invention relate to the system described above, where the controller is further configured to control the cooling element and the heating element to cool or heat a first portion of the epithelium-contacting treatment surface while simultaneously heating or cooling a second portion of the epithelium-contacting treatment surface.

Some embodiments of the invention relate to the system described above, where the epithelium-contacting treatment surface is radially oriented.

Some embodiments of the invention relate to the system described above, where the epithelium-contacting treatment surface has a length between 1 mm and 30 mm and a width between 0.5 cm and 2.0 cm.

Some embodiments of the invention relate to the system described above, where the controller is further configured to activate the cooling element for a first period of time and to activate the heating element for a second period of time. In such embodiments, the first period of time overlaps at least partially with the second period of time.

Some embodiments of the invention relate to the system described above, where the controller is further configured to activate the cooling element for a first period of time and to activate the heating element for a second period of time. In such embodiments, the first period of time commences and ends prior to commencement of the second period of time.

Some embodiments of the invention relate to the system described above, where the cooling element is configured to apply a cooling agent to a surface of the target tissue.

Some embodiments of the invention relate to the system described above, where the cooling agent is selected from the group consisting of compressed liquid N₂, compressed liquid N₂, compressed liquid CO₂, compressed liquid NO₂, a hydrofluorocarbon, water, a thermoelectric cooler and an ultra-low temperature cryogen.

Some embodiments of the invention include an apparatus comprising three parts: a console that controls the therapeutic application of energy, a handpiece that connects to the console, and a treatment tip that attaches to the handpiece and applies the energy to the desired point of therapy on a patient's skin. In such embodiments, the console and handpiece are durable multi-use pieces of equipment. The treatment tip, according to some embodiments, is a onetime use only disposable device. The complete apparatus applies cold therapy to a treatment area. The surface of the treatment tip can have multiple shapes, i.e, rectangular, circular, cylindrical, etc. In some embodiments, the surface area of the treatment tip is approximately 1 square inch. In some embodiments, cold therapy is applied to the area being treated. In some embodiments, the cold therapy is accomplished by evaporating compressed or liquid N₂, CO₂ or NO₂ directed on or near the surface of the treatment tip and then applying the treatment tip to the surface of the tissue by direct contact. The surface of the treatment area is cooled to a desired therapeutic temperature. The temperature is kept within the therapeutic range by applying RF energy via bi-polar electrodes located at the distal end of the treatment tip. The bi-polar electrodes allow the RF energy to heat the treatment area only to a shallow depth—equivalent to the same depth the cold therapy is being applied. The RF energy is throttled in such a way that the treatment area stays within a therapeutic temperature. The therapeutic temperature is established through validation. The therapeutic temperature is low enough to provide positive therapeutic effect but not so low that it ablates the area treated. The system prevents the tissue from falling below the therapeutic temperature creating a cryo-ablation by applying RF energy to warm the skin. The cold therapy triggers the tissue's wound/healing mechanism. That wound/heal mechanism can reduce or eliminate the effect of skin injuries or flaws.

In some embodiments, the controller, including the integrated controller described above, may include a display that is configured to display information about the procedure, the energy and/or heat, the coolant, the treatment tip, the handle and other components of the system. This information may be displayed on the front of the integrated controller, and the controller may present the information with audio signals as well. The display may also be set by the controller to display error information (including error codes) based on the status of the various system components (e.g., coolant level, contact with skin, RF generator status, etc.).

Embodiments relating to the system described above can include a power source. A power source in typical embodiments feeds energy to a heating or cooling source, which heats or cools the treatment tip. For example, RF waves can be produced in a range from 3 kHz to 300 GHz. A multiplexer measures current, voltage and temperature at the thermal sensors associated with each RF electrode. The multiplexer is driven by a controller, which can be a digital or analog controller, or a computer with software. The controller may turn the heating source and cooling source on and off. The controller may determine the length of each cooling and/or heating period in a given “pulse.” The controller may provide multiple different types of pulses that may vary in the duration of cooling or heating. The controller may provide an indication that a pulse has ended, for example, by providing a visual or audio queue. When the controller is a computer it can include a CPU coupled through a system bus. On the system there may also be a keyboard, disk drive, or other non-volatile memory systems, a display, and other peripherals, as are well known in the art. Also coupled to the bus may be a program memory and a data memory.

Some embodiments of the system described above include an operator interface including operator controls and a display. The controller can be coupled to different types of imaging systems including ultrasonic, thermal sensors, and impedance monitors. Current and voltage are used to calculate impedance. A diagnostic phase can be initially run to determine the level of treatment activity. This can be done through ultrasound as well as other means. Diagnostics can be performed both before and after treatment.

In some embodiments of the system described above, the controller is configured to execute a programmed or customizable treatment protocol designed to achieve a predetermined temperature for a predetermined time period in a target tissue to induce remodeling of the target tissue and promote wound healing. The controller instructs the cooling and heating elements to initiate a programmed treatment protocol comprising sequenced pulse duration, pulse timing, and pulse coordinates on a target tissue to induce remodeling of the target tissue and promote wound healing.

One of skill in the art can envision that customizable treatment protocols can be programmed to achieve efficient remodeling and wound healing in specific target tissues. In some embodiments, the treatment protocol includes a plurality of pulses to deliver a cooling agent to the target tissue. These pulses can be spatially overlapping to substantially cover the target treatment area. The extent to which the pulses overlap, as well as the number of pulses used to cover the target tissue area, may depend on the size, location, and number of the cooling and heating element(s), as well as the size, location, and shape of the targeted tissue area.

The method and apparatus, as provided by embodiments of the invention, are non-invasive or minimally invasive and substantially non-ablative of targeted tissues. The nature of the engagement between the apparatus and targeted tissues is that of contacting a treatment tip to a surface region of a target tissue. Through such contact, the apparatus delivers a cooling agent to the surface region, and subsequently cools the target tissue to a therapeutic temperature while preventing ablation. In some embodiments, heat is also applied to assist with the maintenance of the desired therapeutic temperature.

In some embodiments, the cooling mechanism of the apparatus includes a lumen adapted to accommodate a cooling fluid conveyed to nozzles, which cool the cooling element of treatment tip of the probe. Embodiments of the method thus provide for contacting a contact site on a surface of a target tissue using a treatment tip, the treatment tip having the capability both to cool one or more tissue layers of the target tissue and to (optionally) heat the same one or more layers of the target tissue. In some embodiments, the cooling fluid cools the treatment tip of the apparatus, as provided by embodiments of the invention; in turn, the surface of the cooled treatment tip draws energy from the one or more tissue layers of the target tissue that the treatment tip contacts. In some embodiments, the cooling element is configured to apply a cooling agent to a surface of the target tissue. In some embodiments, the cooling agent is selected from the group consisting of compressed liquid N₂, compressed liquid CO₂, compressed liquid NO₂, a hydrofluorocarbon, water, a thermoelectric cooler and an ultra-low temperature cryogen.

As provided by embodiments of the invention, one or more tissue layers of a target tissue may be cooled to a temperature range of about 0.0 degrees Celsius to about 10.0 degrees Celsius, or more preferably to between 1.1 degrees Celsius to about 4.0 degrees Celsius.

In an embodiment of the invention, RF energy is delivered to cooled target tissue. In such an embodiment, RF pulse sequence(s) preceding, following or occurring concurrently with a cooling step serves to protect the cooled one or more tissue layers of the target tissue from ablation as a result of inadvertently cooling the tissue(s) to a temperature below the therapeutic temperature. Importantly, the RF energy heats the same one or more layers of tissue cooled.

In some embodiments of the invention, cooling and maintaining the one or more tissue layers at a therapeutic temperature provokes a cytokine cascade including various heat shock proteins and/or cold shock proteins. This results in remodeling of the target tissue and improvement of the aesthetic or functional defect of the target tissue. In some embodiments, the RF energy is delivered by a bipolar RF energy source.

In some embodiments, the probe and system described above includes a bipolar RF energy heating element. Other embodiments may make use of other forms of energy, such as microwave, laser, or ultrasound.

The energy delivery element may be any of bipolar RF electrodes, a microwave emitter, a laser, or an ultrasound emitter. The RF electrodes, in some embodiments, are capacitive electrodes, which capacitively couple to the mucosal epithelium. The RF electrodes, without limiting the scope of the invention, may have a thickness in the range of about 0.01 to about 1.0 mm. In some embodiments the electrodes can be separated by a predetermined distance. Such a distance can be a function of the depth of tissue penetration desired. In some such embodiments, the electrodes can be separated by a distance between 1 mm to 30 mm, and more preferably between 5 mm and 15 mm.

Additionally, the electrodes may be equipped with an integrated EEROM (Electrically Erasable Read Only Memory, also known as EEPROM) programmable memory chip at any suitable location within the treatment tip (not shown). Such a chip may provide identifying information or other information about the operational status or configuration parameters of the RF electrodes to the system. Such parameters may include, by way of example, the type and size of the electrodes, the number of times the energy delivery element has been fired, and the like. Additionally, thermisters (thermal sensors) may be provided at each corner of the RF electrodes, or otherwise in close proximity to the electrodes, to provide feedback to the system on the temperature at their location.

In some embodiments, RF energy is the preferred energy source over laser or ultrasound. Laser energy can rapidly raise the temperature of the surface layer of tissue, but might not be able to raise the temperature of subsurface tissue layers as effectively and efficiently as RF. In addition, prolonged application of laser energy to a target tissue might cause undesired damage to the surface layer of the tissue, especially if one is applying laser energy with the intent of raising the temperature of subsurface tissue. Although ultrasound might be more effective at heating subsurface tissue layers than RF, it might also cause greater discomfort to the subject leading to premature termination of therapy sessions. As a result, RF is the preferred energy source over ultrasound in some embodiments.

In some embodiments, the cooling element and heating element are positioned on an end of the treatment tip. The cooling element and heating element can have dimensions adapted to making approximately flat contact with the surface of the target tissue. Various lengths, widths, shapes and formations can readily be envisioned and designed to best conform the cooling element and heating element to a specific target tissue.

According to some embodiments of the invention, the treatment surface has a flat configuration. In other embodiments the treatment surface has a radial configuration.

In some embodiments, the treatment tip as a whole is designed as a single-use disposable component, while the hand piece is typically a reusable instrument. The single-use and disposable aspects of the treatment tip are useful in a single procedure in a medical setting.

In some embodiments, the apparatus is included in a larger electronic system (not shown) with features known in the art. Embodiments comprise a power source, a cooling source or energy source that feeds the cooling element, an RF power source that feeds energy to an RF energy generator and energy flows therefrom to RF electrodes. RF waves produced range from 3 kHz to 300 GHz. A multiplexer measures current, voltage and temperature, at the thermal sensors associated with each RF electrode. The multiplexer is driven by a controller, which can be a digital or analog controller, or a computer with software. The controller may turn the cooling source and the RF power source, on and off. The controller may determine the length of each cooling and/or heating period in a given “pulse.” The controller may provide multiple different types of pulses that may vary in the duration of cooling or heating. The controller may provide an indication that a pulse has ended, for example, by providing a visual or audio queue. When the controller is a computer it can include a CPU coupled through a system bus. On the system there may also be a keyboard, disk drive, or other non-volatile memory systems, a display, and other peripherals, as are well known in the art. Also coupled to the bus may be a program memory and a data memory.

An operator interface includes operator controls and a display. The controller can be coupled to different types of imaging systems including ultrasonic transceivers, thermal sensors, and impedance monitors. Current and voltage are used to calculate impedance. A diagnostic phase can be initially run to determine the level of treatment activity. This can be done through ultrasound as well as other means. Diagnostics can be performed both before and after treatment.

Other variations of treatment tip design and associated methods can be employed to achieve the objectives of the invention without departing from the scope of the invention, as will be appreciated by those skilled in the art. The shape and dimensions of the tip can also be adjusted, as desired, to enhance the effectiveness of the treatment taking into consideration physiological and anatomical information. While various embodiments of the present invention have been shown and described herein, it will be obvious to those skilled in the art that such embodiments are provided by way of example only. Although the description has offered the theory that heat shock and/or cold shock protein-mediated responses play a role in tissue remodeling, such discussion has been offered simply as a possible theory of how the invention works and as an aid in describing the invention. It should be understood that any such theories and interpretation do not bind or limit the claims with regard to tissue remodeling brought about by the practice of the invention. Numerous variations, changes, and substitutions will now occur to those skilled in the art without departing from the invention. It should be understood that various alternatives to the embodiments of the invention described herein may be employed in practicing the invention. It is intended that the scope of the invention, methods and structures within the scope of the invention includes equivalents.

Methods

Some embodiments of the invention relate a method for aesthetic treatment, including the steps: non-invasively cooling a surface of a target tissue; and cooling one or more tissue layers of the target tissue to a predetermined therapeutic temperature. The step of non-invasively cooling is performed such that cryoablation of the one or more tissue layers of the target tissue does not occur.

Some embodiments of the invention relate the method above, further including: non-invasively heating the surface of the target tissue; and heating the one or more tissue layers of the target tissue to maintain a temperature of the one or more tissue layers above a temperature at which cryoablation occurs.

Some embodiments of the invention relate the method above, where the non-invasively cooling is performed over a first period of time, the non-invasively heating is performed over a second period of time, and the first period of time overlaps at least partially with the second period of time.

Some embodiments of the invention relate the method above, where the non-invasively cooling commences before the non-invasively heating commences and where the non-invasively heating continues until the non-invasively cooling is terminated.

Some embodiments of the invention relate the method above, where the non-invasively heating occurs concurrently with the non-invasively cooling.

Some embodiments of the invention relate the method above, where the non-invasively heating includes non-invasively applying a heating agent and delivering at least one of radiofrequency energy, microwave energy, laser energy, or ultrasound energy.

Some embodiments of the invention relate the method above where the target tissue includes female genital tissue.

Some embodiments of the invention relate the method above where the target tissue includes tissues of the anus, anal canal and/or rectum.

Some embodiments of the invention relate the method above where the cooling involves cooling the one or more tissue layers to a temperature between 1.1 degrees Celsius and 4.0 degrees Celsius and where the cooling triggers a wound-healing reaction in the one or more tissue layers of the target tissue.

Some embodiments of the invention relate the method above, where the non-invasively cooling includes contacting the one or more tissue layers of the target tissue with a treatment tip during a procedure, the treatment tip including a cooling mechanism.

Some embodiments of the invention relate the method above, further including contacting the one or more tissue layers with the treatment tip at two or more contact sites during the procedure and where the contacting the one or more tissue layers is repeated at least twice during the procedure such that each of the two or more contact sites is contacted at least twice.

Some embodiments of the invention relate the method above, where the non-invasively cooling includes evaporating compressed liquid N₂, CO₂, or NO₂ on a surface of the treatment tip and contacting the surface of the target tissue with the treatment tip.

Some embodiments of the invention relate the method above, where the cooling the one or more tissue layers of the target tissue induces a remodeling of the one or more tissue layers, where the remodeling involves one or more of a release of heat shock proteins and a release of cold shock proteins, and where at least some of the remodeling occurs during the cooling of the one or more tissue layers of the target tissue.

Some embodiments of the invention relate the method above, where the non-invasively applying a cooling agent is done for between 1 second to 300 seconds.

Some embodiments of the invention relate the method above further including the step of treating an aesthetic injury or a functional defect in a subject.

Some embodiments of the invention relate the method above further including the step of treating one or more of a vaginal mucosa, an oral mucosa, a naso-pharyngeal mucosa, an esophageal mucosa, a rectal mucosa or an anal mucosa.

Some embodiments of the invention relate to a method for aesthetic treatment, comprising non-invasively applying a cooling agent to a surface of a target tissue, and cooling one or more tissue layers of the target tissue to a predetermined therapeutic temperature. In such embodiments, applying the cooling agent is performed such that cryoablation of the one or more layers of the target tissue does not occur.

Some embodiments of the invention relate to the method described above, where the one or more tissue layers cooled is a surface tissue layer. It is understood by one of ordinary skill in the art that various types of tissues can be present as surface tissues. The type of tissue is not limited to particular type of tissue or to only one type of tissue. For instance, in some embodiments the surface tissue is a mucosal layer, or an epidermis layer, or a dermis layer.

Some embodiments of the invention relate to the method described above, where a combination of surface tissues are targeted. For instance, in some embodiments multiple surface tissues of the vagina are targeted including the epithelium of the mucosal tissue of the vaginal opening and labium minora (for example) and the epidermis layer or dermis layer of the labia majora. In some embodiments, the various tissues of the anal canal are targeted, including the mucosal tissues of the upper anal canal and the epithelium of the lower anal canal. In some embodiments, tissues of the rectum are targeted. In some embodiments, tissues of the anus are targeted. In some embodiments, the oral mucosa of the mouth is targeted as is the epithelium of the lips outside of the mouth.

Some embodiments of the invention relate to the method described above, where tissue layers exposed due to injury are targeted. For instance, in some embodiments, dermis tissue exposed as a result of injury to an overlying epidermis layer is targeted. Similarly, loose connective tissue in mucosal tissue is targeted in the event of injury to an overlying epithelial layer.

Some embodiments of the invention relate to the method described above, where mucosal tissues are also treated. In such embodiments, the methods include the step of treating one or more of a vaginal mucosa, an oral mucosa, a naso-pharyngeal mucosa, an esophageal mucosa, a rectal mucosa or an anal mucosa.

Some embodiments of the invention relate to the method described above, where the step of cooling one or more tissues includes cooling the one or more tissue layers to a temperature between 0.0 degrees Celsius to about 10.0 degrees Celsius, or more preferably to between 1.1 degrees Celsius to about 4.0 degrees Celsius.

Some embodiments of the invention relate to the method described above, where the step of non-invasively applying a cooling agent includes contacting the one or more tissue layers of the target tissue with a treatment tip during a procedure. In such embodiments, the treatment tip includes a cooling mechanism.

Some embodiments of the invention relate to the method described above, where the step of non-invasively applying a cooling agent includes contacting one or more tissue layers of the target tissue with a treatment tip at two or more contact sites during a procedure. In some embodiments, the step(s) of contacting the one or more tissue layers is repeated at least twice during a procedure such that each of the two or more contact sites is contacted at least twice.

Some embodiments of the invention relate to the method described above, where the step of non-invasively applying a cooling agent includes evaporating compressed liquid N₂, CO₂, or NO₂ on a surface of a treatment tip and contacting the surface tip to one or more tissue layers of a target tissue.

Some embodiments of the invention relate to the method described above, where the cooling agent is liquid N₂, liquid CO₂, liquid NO₂, a hydrofluorocarbon, water, a thermoelectric cooler or an ultra-low temperature cryogen.

Some embodiments of the invention relate to the method described above, where the step of cooling the one or more tissue layers triggers a wound-healing reaction in the one or more tissue layers of the target tissue. In some embodiments, the wound-healing includes the generation of collagen.

Some embodiments of the invention relate to the method described above, where the step of cooling the one or more tissue layers induces a remodeling of the one or more tissue layers. In some embodiments, remodeling includes a release of heat shock and/or cold shock proteins. In some embodiments, at least some of the remodeling occurs during the cooling of the one or more tissue layers of the target tissue.

Some embodiments of the invention relate to the method described above, where the step of non-invasively applying a cooling agent is done for between 1 second to 300 seconds. In some embodiments, the cooling agent is applied continuously for a desired amount of time. In some embodiments, the cooling agent is applied during a sequence of two or more pulses, wherein each pulse is the same duration of time or a different duration of time. In such embodiments, the pulses are separated by a predetermined duration of time.

Some embodiments of the invention relate to the method described above, where the method for aesthetic treatment further includes the step of treating an aesthetic injury or functional defect in a subject.

Some embodiments of the invention relate to the method described above, also including a step of non-invasively applying a heating agent to the surface of the target tissue; and heating the one or more tissue layers of the target tissue to maintain a temperature of the one or more tissue layers above a temperature at which cryoablation occurs.

Some embodiments of the invention relate to the method described above, where the step of non-invasively applying a heating agent is carried out with a probe having a bipolar electrode for applying radiofrequency energy as the heating agent. The applied RF energy creates a band of heat. The heat band depth in the targeted tissue is ½ the distance between the 2 electrodes on the bipolar electrode (conventional)—and that band of heat warms tissue at the given depth and prevents the cold treatment from passing deeper into the tissue. In such embodiments, the cooler surface tissue is located above the band of heat on the surface layer of the tissue. The band of heat serves as a barrier to the cold to prevent the cold from going deeper into the tissue. In such embodiments, different layers of tissue are cooled and heated, with the cooling being done to the surface layer of the tissue and the heating occurring deeper in the tissue below the cold tissue surface layer.

Some embodiments of the invention relate to the method described above, where the step of non-invasively applying a cooling agent is performed over a first period of time, the step of non-invasively applying a heating agent is performed over a second period of time, and the first period of time overlaps at least partially with the second period of time.

Some embodiments of the invention relate to the method described above, where the step of non-invasively applying a cooling agent commences before the step of non-invasively applying a heating agent commences. Also, the step of non-invasively applying the heating agent continues until the step of non-invasively applying a cooling agent is terminated.

Some embodiments of the invention relate to the method described above, where the step of non-invasively applying the heating agent occurs concurrently with the step of non-invasively applying the cooling agent.

Some embodiments of the invention relate to the method described above, where the step of non-invasively applying a heating agent also includes delivering of at least one of radiofrequency energy, microwave energy, laser energy, or ultrasound energy.

Some embodiments of the invention relate to the method described above, where a combination of surface tissues are targeted. For instance, in some embodiments multiple surface tissues of the vagina are targeted including the epithelium of the mucosal tissue of the vaginal opening and labium minora (for example) and the epidermis layer or dermis layer of the labia majora. In some embodiments, the various tissues of the anal canal are targeted, including the mucosal tissues of the upper anal canal and the epithelium of the lower anal canal. In some embodiments, tissues of the rectum are targeted. In some embodiments, tissues of the anus are targeted. In some embodiments, the oral mucosa of the mouth is targeted as is the epithelium of the lips outside of the mouth.

Some embodiments of the invention relate to the method described above, where the target tissue specifically comprises female genital tissue. In some embodiments, individual structures comprising the female genital are targeted. In some embodiments, multiple structures are targeted. Structures comprising the female genital are understood by one of ordinary skill in the art and include, by way of non-limiting example: the clitoral hood, the clitoris, the labium minorum, the vaginal opening, the perineum, and the labia majora.

Some embodiments of the invention specifically relate to methods for aesthetic treatment, comprising non-invasively applying a cooling agent to a surface of a target tissue, where the target tissue is vaginal tissue. In such embodiments, multiple surface tissues of the vagina can be targeted including the epithelium of the mucosal tissue of the vaginal opening and labium minora (for example) and the epidermis layer or dermis layer of the labia majora. Various other tissues and structures within and outside of the vagina can be targeted for therapy.

Some embodiments of the invention include non-invasive treatment of lower portions of the vagina. The lower portions of the vagina are the portions immediately inward from the introitus. An embodiment of the invention provides a non-surgical and non-invasive method for aesthetic treatment. Such a treatment includes non-invasively applying a cooling agent to a surface of a target tissue in at least one lower portion of the vagina and cooling one or more tissue layers of the target tissue to a predetermined therapeutic temperature. In such embodiments, applying the cooling agent is performed such that cryoablation of the one or more layers of the target tissue does not occur. In some embodiments, the target tissue area is inside the vagina directly proximal to the hymenal ring and the cooling of the target tissue induces remodeling of the target tissue. Thus, according to an embodiment of the invention, the portion of the vagina to be treated is a region between the hymen and a position located no further than about 4 to 6 cm inward from the hymen.

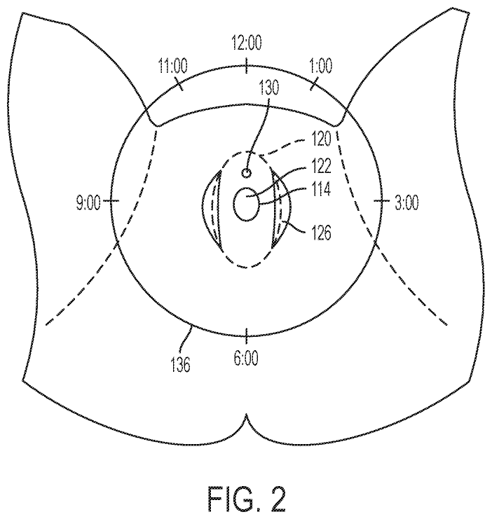

According to an embodiment of the invention, the anatomical areas of the female genitalia treated include the vagina and the introitus, the opening of the vagina. With more specific regard to the vagina, embodiments of the method comprise treating the lower portion of the vagina, a portion extending from the introitus to a location from about 4 cm to about 6 cm inward from the introitus. With regard to the circumference of the inner wall of the vagina, a clock-position reference scheme is helpful. FIG. 2 shows such a schematic, 136. The urethra lies next to the anterior wall of the vagina. Thus, the location of the vaginal wall nearest the urethra and urethral opening may be considered 12 o'clock in FIG. 2.

The vagina is a fibromuscular tube, lined with stratified squamous epithelium that connects the external and internal organs of the female reproductive system. The vagina runs obliquely upwards and backwards at an angle of about 45 degrees between the bladder in front and the rectum and anus behind. In an adult female the anterior wall is about 7.5 cm long and the posterior wall is about 9 cm long. The difference in length is due to the angle of insertion of the cervix through the anterior wall. FIG. 2 is a schematic view of female genitalia depicting the mucosal epithelial surfaces as well as an orienting clock 136 to provide a circumferential reference scheme for the vagina wall. FIG. 2 shows the urethra 130, Hart's line 120, the vaginal opening 122, the introitus 114, and the labium minora 126.

The mucosal epithelium of vulvar tissue outside the vagina and the introitus includes the labia minora, or that portion of the vulva extending outward from the introitus to Hart's line, the boundary where mucosal epithelium and labial skin meet. The mucosal epithelium and the skin, while contiguous, are embryologically and histologically distinct. The portion of the female genitalia that is covered by epithelium is also substantially defined by the bounds of the vestibule, which extends outward or down from the hymenal ring at the top of the vagina, radially beyond the introitus, including the portion of labia minora located within Hart's line 120. The target tissue of some embodiments of this invention include the connective tissue underlying these mucosal epithelial surfaces of the genitalia which, progressing down from the epithelial surface, are known as the lamina propria and the muscularis, respectively. The lamina propria includes a mixture of cell types that populate connective tissue, such as fibroblasts, and the muscularis is a layer of smooth muscle. Collagen is secreted or deposited into the extracellular space in these tissues by cells such as fibroblasts. These described target tissue layers below the epithelium overlay deeper tissues, including endopelvic fascia, which are not a target tissue for embodiments of the present invention.

The method and apparatus, as provided by embodiments of the invention are non-invasive and substantially non-ablative of genital tissue. The nature of the engagement between the apparatus and genital tissue is that of contacting a treatment tip to an epithelial surface of the genital tissue. Through such contact, the apparatus cools one or more layers of a target tissue. In some embodiments, heat is also applied before, during or after the cooling to prevent the tissues from being damaged due to falling below a therapeutic temperature, or below a temperature associated with ablation of the tissue.

According to an embodiment of the invention, the anatomical areas of the human oral and nasal cavities are treated.

According to an embodiment of the invention, the anatomical areas of the human anus, anal canal and/or rectum are treated.

According to some embodiments of the invention, a “pulse” can refer to application of a cooling agent, application of a heating agent and/or simultaneous application of a cooling agent and a heating agent. Some embodiments can include treatment protocols having a variety of pulses applied to a target tissue (e.g., a first pulse for applying a cooling agent followed by a second pulse applying a cooling and a heating agent simultaneously).

In some embodiments where a cooling agent and a heating agent are applied, the cooling agent and the heating agent can be applied simultaneously in one pulse, or applied individually in separate pulses. In embodiments where the cooling agent and the heating agent are applied in separate pulses, the individual pulses can partially overlap in timing of execution such that a first pulse does not terminate before a second pulse commences. In embodiments where the cooling agent and the heating agent are applied in separate pulses where the pulses do not overlap in timing, a “cooling” pulse (i.e. the pulse associated with the application of the cooling agent) may precede or follow a heating pulse (i.e. the pulse associated with application of the heating agent).

The duration of each pulse will vary depending on the nature of the cooling agent or heating agent applied, the type of tissue targeted (as different tissues will require varying amount of time to reach a desired therapeutic temperature), and the duration of time the target tissue is intended to maintain a therapeutic temperature. In general, the duration of a pulse will vary from 0.1 second to 300 seconds. In addition, in some embodiments, a plurality of pulses or varying durations are applied. For example, a procedure may include a first cooling pulse of 1 second, followed by a heating pulse of 1 second, followed by a second cooling pulse of 5 seconds.

According to some embodiments, a procedure may include a period of cooling of the target tissue, followed by a period of rest, and then a second period of cooling. In such embodiments, each “pulse” may include a period of cooling of the target tissue, followed by a period of rest, and then a second period of cooling. The duration of each of the cooling and rest periods may be the same, or may vary.

According to some embodiments, a procedure may include a period of cooling of the target tissue, followed by a period of heating, and then a second period of cooling. In such embodiments, each “pulse” may include a period of cooling of the target tissue, followed by a period of heating, and then a second period of cooling. In some embodiments, the period of heating may at least partially overlap with at least one of the cooling periods, or may entirely overlap with the first or second cooling period. Also, the duration of each of the cooling and heating periods may be the same, or may vary.

The cooling of target tissue, per some embodiments of the invention, includes lowering the temperature of the target tissue to as low as 0.0 degrees C., or to as low as 1.0 degree C., and more preferably as low as 1.1 degree C. The therapeutic temperature in some cases may be only as high as 10.0 degrees C., or as high as 5.0 degrees C., and more preferably only as high as 4.0 degrees C.

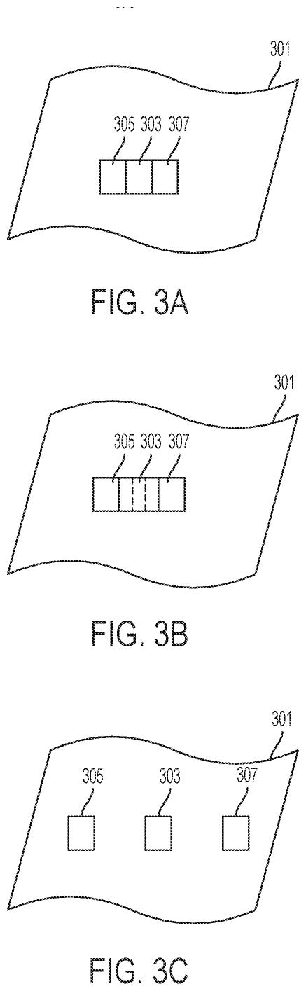

According to an embodiment of the invention, a “pulse” of a cooling agent and/or a heating agent is applied to a plurality of target locations. In such embodiments, a plurality of pulses are delivered to a plurality of contact sites within the target tissue area during a procedure. In such embodiments, the number of pulses delivered varies depending on the surface area of the target area to be treated and the desired area of coverage. An example schematic is depicted in FIG. 3A. In FIG. 3A, for instance, the surface of the target tissue 301 is treated with a first pulse at a first contact site 303 on the target tissue. A second pulse is then delivered to a second contact site 305, the second contact site 305 located at a predetermined distance to the left of the first contact site 303. A third pulse is then delivered to a third contact site 307, the third contact site 307 located at a predetermined distance to the right of the first contact site 303. Additional contact sites can be introduced such that the entire surface area of the target tissue is contacted. In such embodiments, the contact sites can be adjacent to one another (as depicted in FIG. 3A). Alternatively, they can overlap as depicted in FIG. 3B or be spaced apart as depicted in FIG. 3C. One of skill can readily envision that any number of pulses can be applied to a target tissue. In addition, contact sites can be pulsed more than once during a procedure.

In some embodiments of the invention, the treatment protocol each “pulse” includes a first cooling step, a heating step, and a second cooling step. The temperature of the target tissue during the pulse is between 1.1 degree C. and 5 degrees C. The duration of each of the cooling steps is between 1 to 300 seconds, the duration of the heating step is between 1 to 300 seconds. In a system for promoting correction of an aesthetic or functional defect in a target tissue, at least one aesthetic or functional defect treatment parameter is selected to achieve the predetermined temperature and the predetermined time period. For example, the at least one aesthetic or functional defect treatment parameter is selected to achieve a tissue temperature of between 1.1 degrees C. to 5 degrees C. for a time period of 1 to 300 seconds. Example treatment parameters include, but are not limited to the desired and/or therapeutic tissue temperature, the duration the tissue is maintained at the desired and/or therapeutic temperature, the type of cooling agent applied, and the type of heating agent applied. These values are non-limiting example, and aesthetic or functional defect treatment parameters may be selected to achieve other predetermined temperatures for other predetermined time periods in the target tissue to induce remodeling of the target tissue for improvement of the aesthetic or functional defect.

In some embodiments, one or more layers of the target tissue are treated. The total depth of these layers is between 0 mm to 5.0 mm from the surface of the target tissue

In some embodiments, multiple pulses may be administered in sequence, i.e., in a single pass. Alternatively, for some locations, only a single pulse is administered per pass. A pass may include delivering one or more pulses to all of the treatment locations, or to only a subset of the treatment locations. A treatment session may include multiple passes.

In the treatment discussed above, the first heating step protects the target tissue from damage during the cooling step and/or from damage as a result of the tissue temperature from dropping below 1 degree C.

In some embodiments, a procedure, such as would take place in a visit to a medical office, would typically include contacting the surface of the target tissue with a treatment tip on a probe and applying a sequence of pulses. During the same procedure, the treatment tip may be returned to the same contact point multiple times. The total treatment time may be about 30 minutes.

In some embodiments, subsequent treatment(s) can be performed within one month, or at a time later than one month from a first treatment session.

Some embodiments of the method include heating the target tissue using a radiant energy source, typically an RF energy source, but other embodiments may use microwave, ultrasound energy, laser, or magnetic potential energy sources. Some embodiments include contacting the mucosal epithelium with a treatment tip that has an energy delivering element as well as a cooling mechanism.

The method according to some embodiments comprises remodeling of the target tissue. The cooling of one or more tissue layers within the target tissue to a predetermined temperature for a predetermined period of time results an immediate or nearly immediate effect of the activation of heat shock proteins and/or cold shock proteins, resulting in initiation of remodeling of the one or more tissue layers of the target tissue. In other embodiments of the invention, the cooling of the one or more tissue layers during a treatment procedure is understood to result in a subsequent remodeling of the target tissue as part of a biological process that may take place over the course of weeks or months following the procedure.

In another aspect, the apparatus can include three parts: a console that controls the therapeutic application of energy, a handpiece that connects to the console, a treatment tip that attaches to the handpiece and applies the energy to the desired point of therapy on the patient's skin. The console and handpiece can be durable multi-use pieces of equipment. The treatment tip can be a onetime use disposable device. The complete system can apply cold therapy to the treatment area. The surface of the treatment tip can have multiple shapes, i.e, rectangular, circular, cylindrical, etc. The surface area of the treatment tip (for therapy application) can be approximately 1 square inch, for example. Cold therapy can be applied to the area being treated. The cold can be generated by evaporating compressed or liquid N₂, CO₂ or NO₂, directed to the surface of the treatment tip and then applied to the surface of the tissue by direct contact. For example, cryogen can be used to cool the tip or the back of an energy element inside the tip, such as an electrode, and the cool surface of the treatment tip is applied to the surface of the tissue. The treatment area of the tissue can be cooled to the desired therapeutic temperature. The temperature of the treatment area can be kept within the therapeutic range by applying energy, such as RF energy via a bi-polar electrode, at the distal end of the treatment tip. The bi-polar electrode allows the RF energy to heat the treatment area tissue only to a shallow depth, in one aspect, equivalent to the same depth the cold therapy is being applied. The RF energy is throttled in such a way that the treatment area stays within a therapeutic level. The therapeutic temperature level is described herein in accordance with the principles of the invention. The therapeutic level is low enough to provide positive therapeutic effect but not so low that it ablates the area treated. The therapeutic effect is describe herein in accordance with the principles of the invention. The system prevents the cold from falling below the therapeutic level creating a cryo-ablation by using the application of the RF energy to heat and/or warm the tissue. The cold therapy triggers a wound/healing mechanism as described herein. That wound/heal mechanism can reduce or eliminate the effect of skin injuries or flaws.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1.-30. (canceled)

31. A method for treating an aesthetic or functional defect, comprising:
non-invasively cooling a surface of a target tissue;
one or more periods of cooling one or more tissue layers of said target tissue to a predetermined therapeutic temperature; and
one or more periods of rest after the one or more periods of cooling, wherein rest includes not cooling the one or more tissue layers,
wherein said cooling triggers a wound-healing reaction in said one or more tissue layers of said target tissue and is performed such that cryoablation of said one or more tissue layers of said target tissue does not occur.

32. The method of claim 31, further comprising two or more periods of cooling, the one or more periods of rest occurring between two of the two or more periods of cooling.

33. The method of claim 31, further comprising maintaining the predetermined therapeutic temperature by heating said one or more tissue layers of said target tissue.

34. The method of claim 33, wherein only said cooling triggers a wound-healing reaction in said one or more tissue layers of said target tissue.

35. The method of claim 31, wherein no heating is applied.

36. The method of claim 31, further comprising maintaining the predetermined therapeutic temperature without heating said one or more tissue layers of said target tissue.

37. The method of claim 31, wherein the one or more periods of cooling further comprises a first period of time and a third period of time and the one or more periods of rest comprises a second period of time, the method further comprising:
cooling, for the first period of time, the one or more tissue layers of said target tissue to the predetermined therapeutic temperature,
resting, for the second period of time, and
cooling, for the third period of time, the one or more tissue layers of said target tissue.

38. The method of claim 37, wherein the first period of time, the second period of time, and the third period of time are the same.

39. The method of claim 37, wherein the first period of time, the second period of time, and the third period of time vary.

40. The method of claim 37, further comprising non-invasively heating the target tissue during the second period of time.

41. The method of claim 40, wherein the heating at least partially overlaps with the first period of time, the third period of time, or both.

42. The method of claim 40, wherein the heating entirely overlaps with the first period of time, the third period of time, or both.

43. The method of claim 40, wherein said non-invasively cooling commences before said non-invasively heating commences and wherein said non-invasively heating continues until said non-invasively cooling is terminated.

44. The method of claim 40, wherein said non-invasively heating occurs concurrently with said non-invasively cooling.

45. The method of claim 40, wherein said non-invasively heating comprises non-invasively applying a heating agent and delivering at least one of radiofrequency energy, microwave energy, laser energy, or ultrasound energy.

46. The method of claim 31, wherein said cooling comprises cooling said one or more tissue layers to a temperature between 1.1 degrees Celsius and 4.0 degrees Celsius.

47. The method of claim 31, wherein said cooling comprises cooling said one or more tissues layers to a temperature between 0.0 degrees Celsius and 10.0 degrees Celsius.

48. The method of claim 31, wherein said non-invasively cooling comprises contacting said one or more tissue layers of said target tissue with a treatment tip during a procedure, the treatment tip including a cooling mechanism.

49. The method of claim 48, further comprising contacting said one or more tissue layers with said treatment tip at two or more contact sites during said procedure and wherein said contacting said one or more tissue layers is repeated at least twice during said procedure such that each of said two or more contact sites is contacted at least twice.

50. The method of claim 48, wherein said non-invasively cooling comprises applying a cooling agent to the treatment tip, wherein the cooling agent is one or more of compressed liquid N₂, compressed liquid CO₂, compressed liquid NO₂, a hydrofluorocarbon, water, a chemical refrigerant, a cryogen, a thermoelectric cooler and an ultra-low temperature cryogen.

51. The method according to claim 31, wherein said cooling said one or more tissue layers of said target tissue induces a remodeling of the one or more tissue layers, wherein said remodeling comprises one or more of a release of heat shock proteins and a release of cold shock proteins, and wherein at least some of said remodeling occurs during the cooling of said one or more tissue layers of said target tissue.

52. The method according to claim 31, wherein said non-invasively applying a cooling agent is done for between 1 second to 300 seconds.

53. The method of claim 31, further comprising treating one or more of a vaginal mucosa, an oral mucosa, a naso-pharyngeal mucosa, an esophageal mucosa, a rectal mucosa, an anal mucosa, promoting correction of an aesthetic defect, or promoting correction of a functional defect.

54. A device for cooling a target tissue, the device comprising:
a probe; and
a treatment surface located on the probe and divided into a plurality of sections along a length of the probe, each of the plurality of sections including one or more energy delivery elements configured to heat, cool, or both heat and cool the target tissue,
wherein a first section of the plurality of sections heats the target tissue simultaneously with a second section of the plurality of sections cooling the target tissue.

55. The device of claim 54, wherein the one or more energy delivery elements comprises one or more cooling elements, and
wherein the one or more cooling elements are configured to cool the target tissue and trigger a wound-healing reaction in the target tissue.

56. The device of claim 54, wherein the one or more energy delivery elements comprises one or more heating elements.

57. The device of claim 54, wherein the one or more energy delivery elements further comprise a plurality of cooling elements and a plurality of heating elements, wherein the plurality of cooling elements and the plurality of heating elements are configured to cool and heat, respectively, the plurality of sections of the treatment surface separately, in succession, or both.

58. The device of claim 54, wherein the one or more energy delivery elements are configured to successively cool the treatment surface from the first section of the plurality of sections to a last section of the plurality of sections.

59. The device of claim 58, wherein the cooling of each section of the plurality of sections does not overlap in time.

60. The device of claim 58, wherein the cooling of each section of the plurality of sections is individually controlled.

61. The device of claim 58, wherein the one or more energy delivery elements are configured to successively heat the treatment surface from the first section of the plurality of sections to the last section of the plurality of sections after a respective section of the plurality of sections is cooled.

62. The device of claim 54, further comprising a plurality of treatment surfaces and an adjustment mechanism configured to move the plurality of treatment surfaces closer together, farther apart, or adjacent to one another to form a continuous treatment surface.

63. The device of claim 54, further comprising a plurality of treatment surfaces, wherein the plurality of treatment surfaces are parallel to one another.

64. The device claim 54, further comprising a plurality of treatment surfaces, wherein the plurality treatment surfaces are angled with respect to one another and to the probe.
